(19) United States
(12) Patent Application Publication
(10) Pub. No.: US 2022/0226531 A1
(43) Pub. Date: Jul. 21, 2022

(54) UVC DURABLE FILTER

(71) Applicant: Crystal IS, Inc., Green Island, NY (US)

(72) Inventor: Christopher Scully, Troy, NY (US)

(21) Appl. No.: 17/576,427

(22) Filed: Jan. 14, 2022

Related U.S. Application Data
(60) Provisional application No. 63/138,121, filed on Jan. 15, 2021.

(51) Int. Cl. A61L 9/20; A62B 18/02

(52) U.S. Cl. CPC A61L 9/20; A62B 18/025; A61L 2209/16; A61L 2209/12; A61L 2209/14

(57) ABSTRACT

A system includes a mask having a filter. The filter is configured to capture aerosol particles. The filter may be formed from a material that is UVC durable and UVC transmissive. The system also includes a UVC LED configured to emit UVC radiation into the filter.

### PRIORITY

This patent application claims priority from provisional U.S. patent application No. 63/138,121, filed Jan. 15, 2021, entitled, “UVC DURABLE FILTER,” and naming Christopher Scully as inventor, the disclosure of which is incorporated herein, in its entirety, by reference.

### FIELD OF THE INVENTION

Illustrative embodiments of the invention generally relate to treating filters by UV disinfection and, more particularly, illustrative embodiments relate to a facemask containing the filter.

### BACKGROUND OF THE INVENTION

UVC light penetrates the cells of microorganisms and disrupts the structure of their DNA and RNA. This disruption prevents the microorganism from surviving and/or reproducing, rendering it inactive and no longer pathogenic.

### SUMMARY OF VARIOUS EMBODIMENTS

In accordance with one embodiment of the invention, a system includes a filter configured to capture aerosol particles. The filter may be formed from a material that is UVC durable and UVC transmissive. The system also includes a UVC LED configured to emit UVC radiation into the filter.

In various embodiments, the filter is formed from a plurality of fibers. The filter may be integrated into a mask. Additionally, or alternatively, the filter may also be integrated into an HVAC system, a portable air cleaning device, an air circulation system of an aircraft, a vehicle, or an elevator.

Some embodiments may include a plurality of UVC LEDs and/or a plurality of filters. The LED may a lidless type LED. Some embodiments may include an optical coupler on an exposed surface of the LED (e.g., on the LED die). The UVC LEDs may be integrated into the mask. Furthermore, the mask may include a UVC blocker configured to protect a portion of a user's face from UVC. The UVC blocker may include UVC goggles 24. The UVC blocker may be formed of a UVC durable and/or UVC absorbent material. Furthermore, the UVC blocker may be formed by contoured portions of the mask and/or a separate component.

In various embodiments, the mask is a respirator, an N95 mask, or a cloth mask. The filter may be formed from at least one of PTFE, PET, and glass. Furthermore, the filter may be part of a replaceable cartridge for the mask.

In accordance with another embodiment, an apparatus includes a filter formed from a material that is UVC transmissive and UVC durable. The filter has pores through which air may travel. The filter is configured so that radiation from a UVC LED irradiates the entirety of the filter to inactivate pathogens captured by the filter.

In various embodiments, the system is configured to periodically dose UVC. To that end a controller may be configured to set a duty cycle for the period. The duty cycle is a ratio of the active duration to the period. In various embodiments, the duty cycle is less than or equal to about 1:100, and greater than or equal to about 1:5760. In various embodiments, the active duration may be between about 10 seconds and about 5 minutes. Furthermore, the period may be between about 30 minutes and about 48 hours.

In accordance with another embodiment, a method filters air. A filter configured to capture aerosol particles is provided. The filter is formed of a material that is UVC durable and UVC transmissive. A UVC LED configured to emit UVC radiation into the filter is also provided. A pathogen is captured using the filter. The filter is radiated/dosed with UVC light to disinfect the filter.

In various embodiments, the filter is configured so that UVC light reaches all portions of the filter. The filter may be at least 90% UVC transmissive. The UVC light is periodically dosed. In some embodiments the light is dosed for less than 1 second. In some embodiments the UVC light is dosed for at least 1 second, at least 2 seconds, at least 5 seconds, at least 10 seconds, or at least 20 seconds. In various embodiments, the filter may be dosed with at least 5 mJ/cm² of UVC. In various embodiments, the filter is configured to be dosed with UVC for at least 1000 hours without degrading. The the pathogen captured in the filter may be disinfected by at least 1 log reduction value (i.e. 90% reduction).

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows people wearing various masks in accordance with illustrative embodiments.

FIG. 2 schematically shows a magnified view of the fibers of a cotton mask compared with aerosol particles of different sizes in accordance with illustrative embodiments.

FIGS. 3A-3D schematically show a variety of masks including the filter and the LED in accordance with illustrative embodiments of the invention.

FIGS. 4A-5B schematically show details of LEDs that may be used in accordance with illustrative embodiments of the invention.

FIG. 6 schematically shows a method of using the filter in accordance with illustrative embodiments.

FIG. 7 schematically shows the filter in use with a cabin of a commercial jet aircraft in accordance with illustrative embodiments of the invention.

FIG. 8 schematically shows the filter in use with a UVC photoreactor in accordance with illustrative embodiments of the invention.

FIGS. 9A-9E schematically show the filter in various use scenarios in accordance with illustrative embodiments of the invention.

FIG. 10A schematically shows a periodic dosing protocol in accordance with illustrative embodiments of the invention.

FIG. 10B schematically shows a dosing schedule with periods based on expiration of the last scheduled UVC dose in accordance with illustrative embodiments of the invention.

FIG. 10C schematically shows an adjusted dosing schedule with periods based on expiration of the last UVC dose in accordance with illustrative embodiments of the invention.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

In illustrative embodiments, a respirator or mask (both generally referred to herein as “mask”) includes a filter configured to trap pathogens, such as novel coronavirus SARS-CoV-2. The mask includes a UVC light emitting diode (LED) that may be integrated into the mask and/or the filter. The LED is configured to provide UVC light to the filter. The filter and/or various parts of the mask (e.g., mask-filter interface) are UVC durable and UVC transmissive. Furthermore, in some embodiments, the material of the filter is UVC diffusively reflective. Details of illustrative embodiments are discussed below.

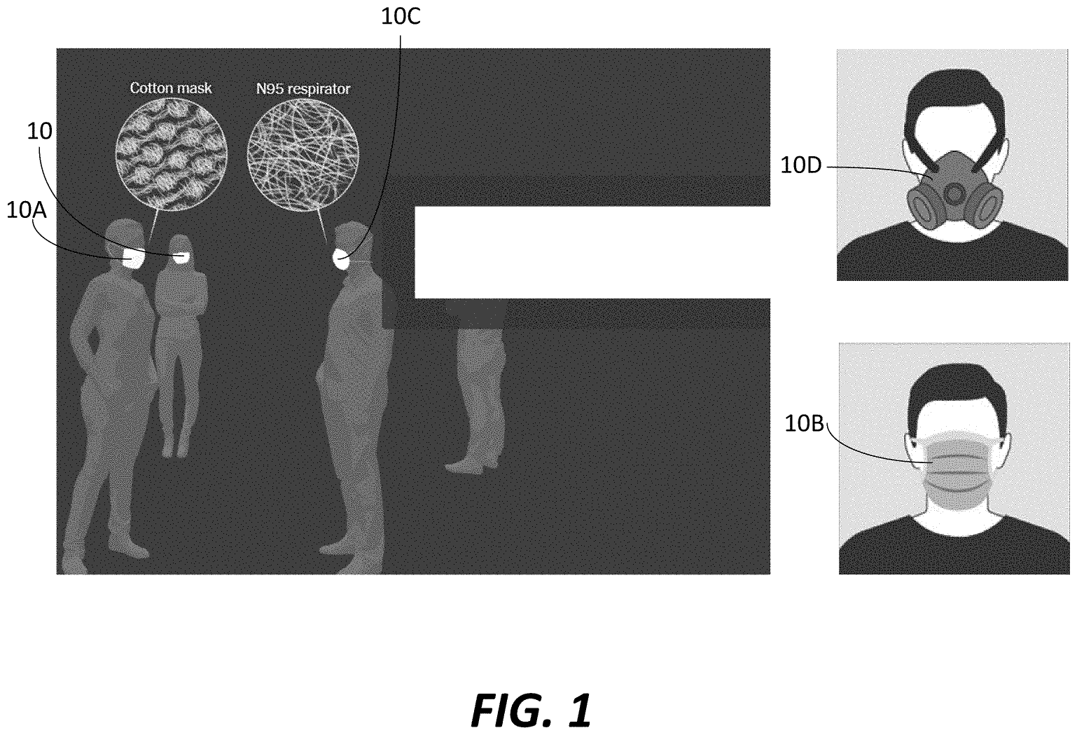

FIG. 1 schematically shows people wearing various masks 10 in accordance with illustrative embodiments. Given the current outbreak of Coronavirus Disease 2019 (COVID-19) caused by the novel coronavirus SARS-CoV-2, consumers frequently wear masks 10 of the type shown in FIG. 1. The masks shown in FIG. 1 include a cotton mask 10A, a disposable mask 10B, an N95 mask 10C, and/or an elastomeric mask 10D. Collectively, these various types of masks are referred to as masks 10. Current “filters” for masks 10 include tight woven cotton masks, or synthetic non-wovens.

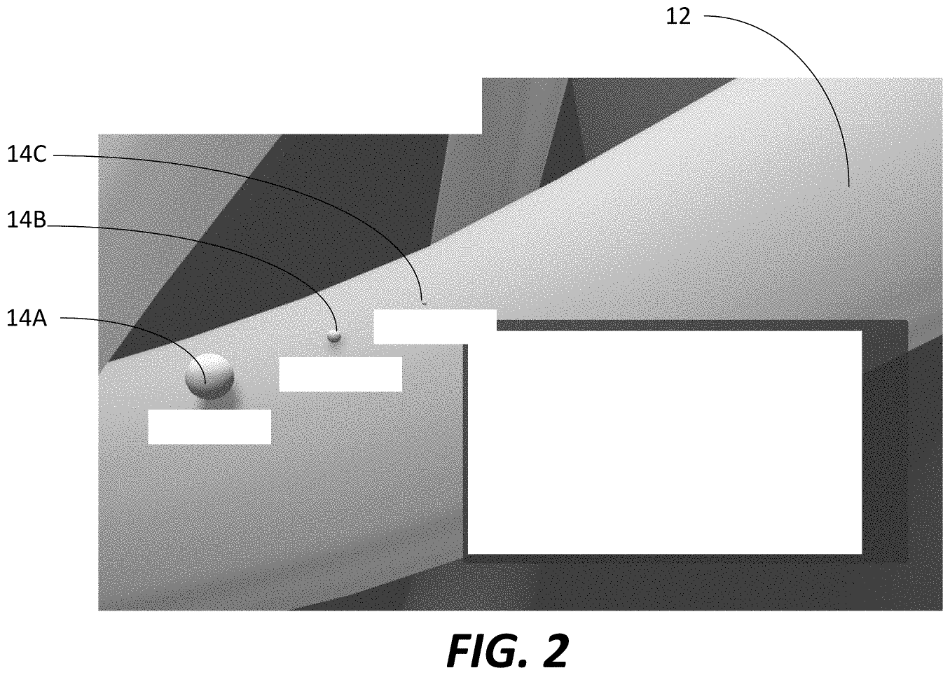

FIG. 2 schematically shows a magnified view of the fibers 12 of a cotton mask 10 compared with aerosol particles 14 of different sizes 14A-14C in accordance with illustrative embodiments. The larger particles 14A in FIG. 2 are greater than 0.5 microns in diameter. Medium particles 14B are about 0.1 micron to about 0.5 micron diameter. Small particles 14C can be less than 0.1 microns in diameter. The coronavirus is about the size of the smallest particles 14C, but it usually travels inside the larger particles 14A and 14B.

With current filters, most of the aerosols in the air are filtered out. However, filters become clogged with moisture, eventually evaporating or pushing aerosols through the filter. Furthermore, prior art filters cannot easily be decontaminated with UVC, as they are typically opaque to UVC, and also vulnerable to degradation by UVC, leading to ineffective decontamination and shortened filter lifespans. Thus, the filters themselves disadvantageously provide areas where the pathogens can hide from UVC radiation. Accordingly, in such filters, UVC does not kill all of the pathogens.

Disposable facemasks 10B, such as surgical or medical masks, are not respirators and do not protect the wearer from breathing in small particles 14C, gases, or chemicals in the air. Disposable facemasks 10B act as a protective barrier to prevent splashes, sprays, large droplets, or splatter from entering the wearer's mouth and nose. The protective quality of disposable facemasks 10B varies depending on type of material used to form the facemask. Disposable facemasks 10B also help prevent the wearer from spreading respiratory droplets. Because disposable facemasks 10B help prevent the wearer from spreading respiratory droplets, they may slow the spread of the virus that causes COVID-19. Thus, wearing the disposable facemasks 10B may help people who unknowingly have the virus from spreading it to others.

Cloth face coverings are only intended to help contain the wearer's respiratory droplets from being spread. Used in this way, CDC has recommended cloth face coverings to slow the spread of the virus that causes COVID-19. Wearing them may help people who unknowingly have the virus from spreading it to others.

The virus is transmitted by water droplets (aerosol) smaller than the pore size of these filters, but many layers of fibers 12 trap most of the water droplets in the filter. Illustrative embodiments advantageously disinfect the filter and/or the mask 10 using ultraviolet-C(UVC) lamps or LEDs. UVC radiation has been shown to destroy the outer protein coating of the SARS-Coronavirus, which is a different virus from the current SARS-CoV-2 virus. The destruction ultimately leads to inactivation of the virus. The inventors believe that UVC radiation also effectively destroys SARS-CoV-2.

Illustrative embodiments may inactivate a variety of different pathogens, including a variety of viruses. The inventors believe that UVC radiation of various wavelengths effectively inactivates the SARS-CoV-2 virus. Generally, UVC cannot inactivate a virus or bacterium if it is not directly exposed to UVC. In other words, the virus or bacterium is not inactivated if it is covered by dust or soil, embedded in porous surface or on the underside of a radiated surface if the material of the surface is opaque to UVC. It should be understood that illustrative embodiments inactivate a variety of different viruses, including the SARS-CoV-2 virus and mutations thereof.

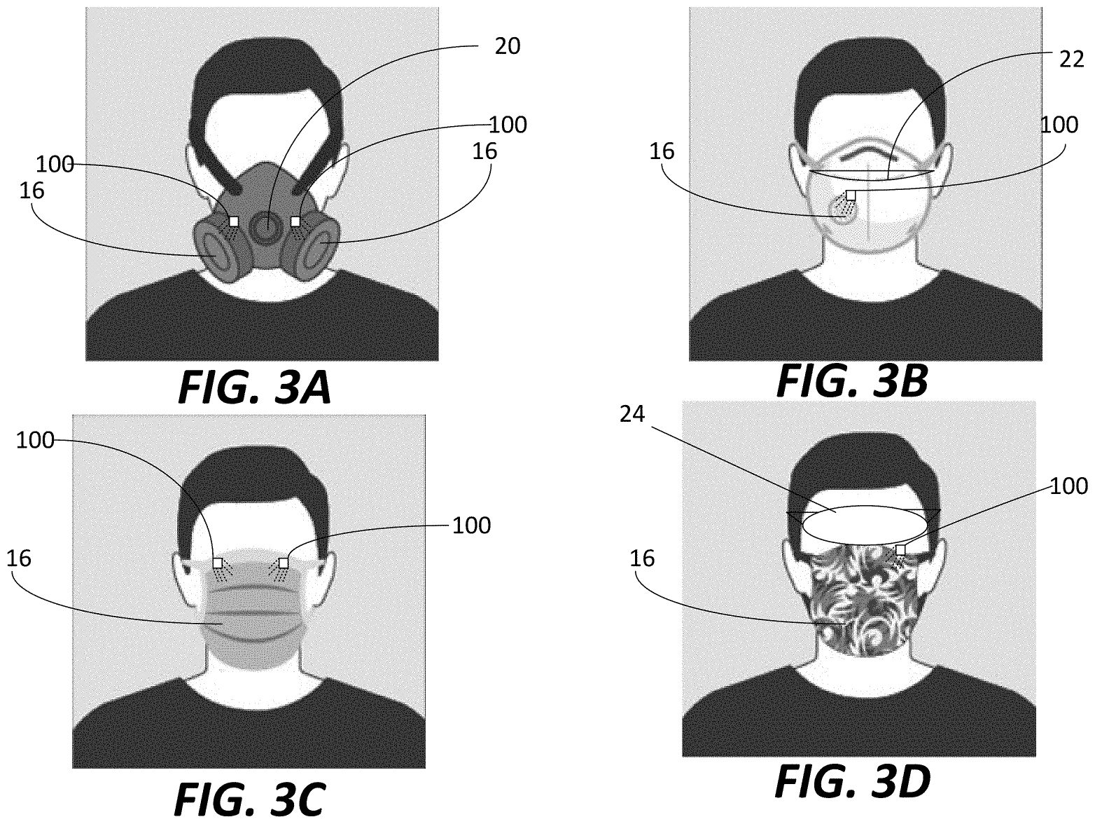

FIGS. 3A-3D schematically show a variety of masks 10 including the filter 16 and the LED 100 in accordance with illustrative embodiments of the invention. Although reference is made to a single LED 100, it should be understood that reference to a single LED 100 may also include a plurality of LEDs 100. Illustrative embodiments form the filter 16 that captures the virus/pathogen from material that is both UVC transmissive and UVC diffusively reflective. For example, the filter may be formed from a Polytetrafluoroethylene (PTFE) weave or non-woven material. In some embodiments, such as in FIG. 3C, substantially all of the mask 10 may be formed by the filter 16.

In some embodiments (e.g., shown in FIGS. 3A-3B), the filter 16 is a small portion of the overall mask 10 (i.e., a respirator style mask 10). However, in some other embodiments (e.g., shown in FIGS. 3C-3D), the majority of the mask 10 may be formed of the filter 16 (e.g., cloth mask). Regardless of the type of the mask 10, the mask may include the LED 100 (e.g., integrated or separate component).

In illustrative embodiments, the filter 16 is formed of a breathable material that includes a tight weave configured to effectively stop/trap aerosols. The material is preferably UVC durable, so that it does not degrade under repeated or continuous UVC exposure (e.g., able to withstand 1000s of hours of high intensity UVC). For example, in various embodiments, the filter 16 (e.g., the UVC durable material) may be exposed to 20 mJ/cm² of UVC for more than 1000 hours with degrading. Furthermore, as described above, the filter 16 material is preferably UVC transmissive to effectively dose UVC through the entire filter 16. If the material is both transmissive and diffusely reflective, like a hypothetical PTFE weave or non-woven material, the filter 16 would “glow” with UVC radiation upon being irradiated from the LED 100 (or other UVC source), ensuring distribution of the radiation throughout the filter 16. In some embodiments, the UVC filter 16 may be formed from a material that is greater than 60% UVC transmissive, greater than 80% UVC transmissive, or preferably, greater than 90% UVC transmissive. Preferably, the filter 16 is formed from a material that is transmissive enough to allow UVC photons to disinfect the whole filter 16 depth.

In illustrative embodiments, the filter 16 may be formed from PET, PTFE, and some types of glass having the noted diffusively reflective and/or transmissive qualities. PTFE is both durable to UVC, diffusively reflective to UVC, and UVC transmissive at thicknesses appropriate for an air filter 16. PTFE filter 16 membranes can be air-restrictive. Therefore, some embodiments may include an exhalation valve 20 (e.g., flapper valve or other one-way valve) to make breathing newly filtered air easier.

In various embodiments, the filter 16 may be housed within a replaceable cartridge (e.g., for use with masks of the type shown in FIG. 3A-3B). To that end, the filter may be disinfected by UVC while part of the mask 10, as part of the filter 16 itself, or by LED 100 applied to the filter 16 after it is separately removed. Furthermore, illustrative embodiments may advantageously dose the LED 100 continuously or periodically. This decontaminates the air breathed by the wearer more than a photoreactor or filter 16 alone.

Although various embodiments show LEDs 100 on the surface of the various masks 10, it should be understood that in some embodiments at least some portion (or all) of the LED 100 may be built into the mask 10 and/or hidden from the views shown in FIGS. 3A-3D. For example, as noted above, the LED 100 may be built into a replaceable cartridge (e.g., in FIG. 3A). Furthermore, the LED may be battery operated. In some embodiments, the LED 100 may also have a wireless power receive coil, and may be wirelessly charged.

In some embodiments, the mask 10 may include a UVC blocker 22 (e.g., UVC durable absorbent) that is configured to prevent UVC radiation from reaching parts of the user's face (e.g., eyes, skin, lips, nose, ears, etc.). In some other embodiments, a disinfection system may include the mask, the filter, and a UVC blocker such as UVC eye-protection goggles 24. The UVC blocker 22 may be a separate piece that projects out (e.g. FIG. 3B) and absorbs UVC radiation that may otherwise contact the wearer's face. Additionally, or alternatively, the mask 10 itself may be contoured to block UVC from reaching the wearer's face (e.g., recessed portion for LED 100 with elevated surrounding portion that blocks radiation pathway to wearer's face). To that end, some portions of the mask 10 (e.g., non-filter portions) may be formed from UVC absorbent material. Furthermore, in some embodiments, the mask 10 may include thermal insulation configured to protect the wearer's face from heat generated by the LED 100.

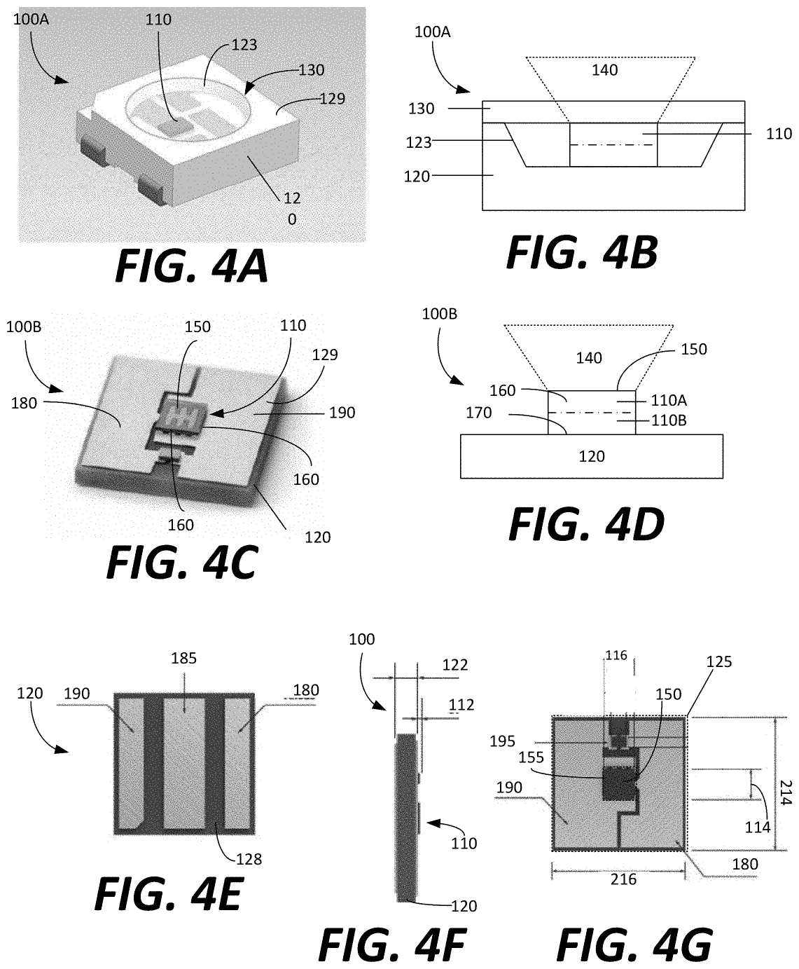

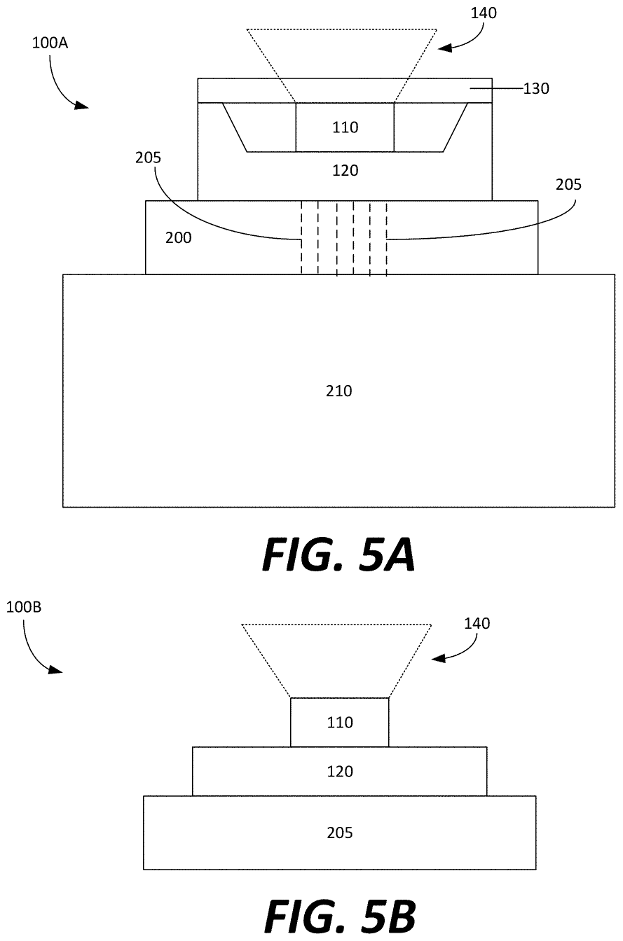

FIGS. 4A-5B schematically show details of LEDs 100 that may be used in accordance with illustrative embodiments of the invention. The light-emitting diodes 100 that produce UV radiation are becoming more commonly available. As used herein, the term “LED” refers collectively to an LED chip 110 and a package 120. To the inventors' knowledge, there are two types of surface mounted UV LEDs 100. The first type of LED 100A (shown in FIGS. 4A-4B) includes the LED chip 110, the package 120 containing the LED chip 110, and a lens 130 (e.g., a quartz window 130) covering the LED chip 110 within the package 120. The second type of LED 100B (shown in FIGS. 4C-4G) includes an exposed LED chip 110 and a package 120 (e.g., a lidless package) containing the LED chip 110. The second type of LED 100B may be a commercially available device, such as the KLARAN™ UV LED, distributed by Crystal IS, Inc. and Asahi Kasei. Furthermore, various embodiments may include an optical coupler 30 configured to increase the effective dosage of radiation emitted by the LED. These LEDs and the optical coupler 30 are described in greater detail in copending U.S. application Ser. No. 17/115,737, incorporated herein by reference in its entirety.

Typically, UVC LEDs 100 emit a very narrow wavelength band of radiation. Currently available UVC LEDs 100 have peak wavelengths at 214 nm, 265 nm, and 273 nm, among others. One advantage of LEDs 100 over low-pressure mercury lamps is that they contain no mercury. Because LEDs 100 have smaller surface area and higher directionality, they may be considered less effective for germicidal applications.

However, illustrative embodiments advantageously form the filter 16 from materials, such as PTFE, that are both UVC transmissive and UVC diffusively reflective. The properties of these materials allow smaller-dosage UVC radiation (as compared to mercury lamps of similar size and weight) to spread throughout the filter 16 (e.g., throughout the fibers 12) thereby inactivating various pathogens trapped in the filter 16. In some embodiments, the filter may be formed from a porous PTFE material that could be made substantially transparent to UVC radiation.

Prior art filters that are not UVC transmissive and/or UVC diffusively reflective may not achieve high levels of pathogen inactivation. In some embodiments, the filter 16 may be formed of a material that is UV translucent (e.g., some of the light would transmit through the filter and some would be reflected). For example, forming the filter 16 of a material that is at least partially UVC transmissive allows the UVC radiation to pass through the material of the filter 16. Furthermore, forming the filter 16 of a material that is at least partially UVC diffusively reflective allows the UVC radiation to reach all, or substantially all, of the pathogens trapped in the filter 16. A material having a combination of these features (i.e., UVC transmissive and UVC diffusively reflective) advantageously provides both benefits.

The UV LED chip 110, also referred to as the UV LED die 110, may be formed of a plurality of semiconductor layers 110A and 110B (e.g., sapphire on GaAlN). The two semiconductor layers 110A and 110B (see FIG. 4D) are shown for convenience only. A person of skill in the art understands that LED chips 110 may be formed of many more layers than those shown. In illustrative embodiments, the LED chip 110 is formed with an aluminum nitride (AlN) substrate having one or more quantum wells and/or strained layers, including AlN, gallium nitride (GaN), indium nitride (InN), or binary or tertiary alloy thereof. The LED chip 110 preferably has a substrate and/or device structure resembling those detailed in U.S. Pat. No. 7,638,346, filed on Aug. 14, 2006, U.S. Pat. No. 8,080,833, filed on Apr. 21, 2010, and/or U.S. Patent Application Publication No. 2014/0264263, filed on Mar. 13, 2014, the disclosures of which are incorporated herein, in their entireties, by reference. As known to those skilled in the art, the specific semiconductor materials and layer structure of the light emitting diode 100 may be selected so that a desired specific wavelength (or wavelength range) of light is emitted by the LED 100. Preferably, the LED chip 110 emits UV light having a peak wavelength range of 260 nm to 270 nm to provide effective, consistent treatment of the filter.

The LED chip 110 has a top radiation emission surface 150 from which UV light is emitted. However, while most of the UV light is emitted from the top surface 150 (also referred to as top light emitting surface 150), some smaller portion of the UV light may also be emitted by side surfaces 160 of the LED chip 110. Therefore, in illustrative embodiments, the top surface 150 is considered the primary light emitting portion and the side surfaces 160 may be considered non-primary light emitting portions of the LED 100.

To capture some of the side emitted light, some embodiments provide, on or in the package 120, a reflective inwardly facing surface 123 configured to reflect UV light emitted by the side surfaces 160. Reference to the “light emitting surface 150” and/or “light emission surface 150” is generally intended to refer to the primary light emitting portion of the LED 100. Furthermore, illustrative embodiments should not be interpreted as requiring a planar “surface” for the primary light emitting surface 150 of the LED 100, although some embodiments can have a planar surface for that purpose.

The bottom surface 170 of the LED chip 110 may be electrically and thermally coupled with the top surface of the package 120. As known to those of skill in the art, the LED 100 may include electrical contacts such as an anode 180 and a cathode 190. For example, as shown in FIG. 4C, the LED chip 110 may be mounted directly on the cathode 190. Such contacts may electrically couple to the chip 110 through the thickness of the package 120, e.g., using one or more vias or other connectors within the package 120.

FIG. 4E schematically shows a bottom surface 128 of the package 120, having the cathode 190, a thermal plate 185, and the anode 180. In use, the LED 100 is surface mounted on a printed circuit board (e.g., a flexible PCB). The mask 10 may include a slot for positioning of the printed circuit board, such that the printed circuit board does not contact a user's face. The LED 100 may be electrically coupled with the PCB 200 through the anode 180 and the cathode 190. Furthermore, to spread heat, the LED 100 may be conductively thermally coupled with the PCB 200 by means of above noted thermal plate 185. To augment the thermal plate 185, the package 120 preferably also has a thin form factor, and/or is made from a material having a low thermal resistance.

FIGS. 4F and 4G schematically show side and top views of the LED 100 of FIG. 4E, respectively. In some embodiments, the package 120 may have a thickness or height 122 of about 0.50 millimeters, a length 124 of about 3.50 millimeters, and a width 126 of about 3.50 millimeters. The LED chip 110 has dimensions that may be relatively small compared to the package 120. For example, the chip 110 may have a thickness or height 112 of about 0.11 millimeters, a length 114 of about 0.80 millimeters, and a width 116 of about 0.80 millimeters.

While the discussion of dimensions refers to LED 100B, it should be understood that illustrative embodiments are not limited to the dimensions described herein. Furthermore, the LED 100A may have the same or similar dimensions for the same or similar components.

The top surface 150 may define an area 155 or a perimeter 155. Generally, as noted above, the top surface 150 of an LED chip 110 may be substantially planar. However, illustrative embodiments may texture the top surface 150 and/or shape the top surface 150 in some other manner, such as in a “V” shape. Therefore, the top surface area 155 is intended to cover the area defined by the outer bounds and/or a perimeter of the top surface 150. In such an instance, the area 155 is not intended to be calculated by adding together the various portions that form the “top surface.” For example, if the top surface 150 is a combination of two surfaces forming a “V” shape, the area 155 is the perimeter defined by the outer bounds of the “V” shaped top surface, and not the sum of the two separate surface areas. Accordingly, unless the context suggests otherwise, the area 155 is defined by the perimeter of the top surface 150.

FIG. 5A schematically shows the LED 100A of FIGS. 4A-4B mounted on a printed circuit board 200 and a heat sink 210, in accordance with illustrative embodiments of the invention. When the LED 100A emits UV light (e.g., represented by cone of radiation 140), it can produce a considerable amount of heat. Undesirably, excess heat negatively impacts the light output and lifetime of the LED 100. Thus, proper thermal management preferably keeps the junction temperature (TJ) as low as is required for the given application and maintains the performance of the LED. The word “junction” refers to the p-n junction within the LED die 110, where the photons are generated and emitted. As shown in FIG. 5A, heat may be transferred away from this junction to the ambient by attaching the heat sink 210. To further assist with heat transfer, the PCB 200 may include thermal vias 205. Preferably, in illustrative embodiments the heat sink 210 is positioned so as not to contact the user's face. Furthermore, the heat sink is preferably covered to protect inadvertent contact with the heat sink 210. For short duration pulse operation of the LEDs 100, illustrative embodiments may mount the LED on metal core PCB. Additionally, air flow aids in the cooling of the MCPCB.

FIG. 3B schematically shows the LED 100B of FIGS. 4C-4F mounted on the printed circuit board 200. In some embodiments, including that of FIG. 5A, the LED 100 is not coupled with the heat sink 210. Instead, because the LED chip 110 is exposed (e.g., the top light emitting surface 150 is not covered), illustrative embodiments may contact the LED chip 110 with an optical coupler having a low thermal resistance (as shown in FIG. 5, discussed below). The optical coupler draws heat away from the LED 100B, and allows for effective thermal management without the addition of the heat sink 210. Accordingly, some embodiments may have a considerably smaller form factor than may otherwise be available with the use of the heat sink 210. It should be understood, however, that some other embodiments may include the heat sink 210.

The figures schematically show the light emitting diode 100 on the substrate package 120. Among other things, this schematic drawing may represent one or more UV light emitting diodes 100, as well as supporting electronics, such as voltage regulators, avalanche breakdown diodes, silicon-controlled rectifiers, Zener diodes, and power sources. The package 120 may include one or more plastics, such as polyphthalamide (PPA) and/or one or more ceramics, such as aluminum nitride and/or alumina. In various embodiments, as noted above, one or more portions of a surface of the package 120 may be coated with a material reflective to UV light (e.g., aluminum or PTFE) and/or that is electrically and/or thermally conductive (e.g., one or more metals).

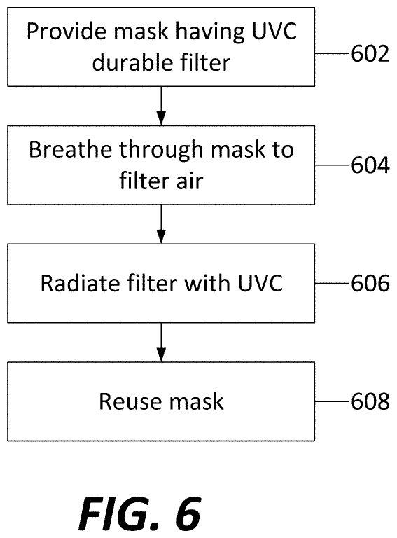

FIG. 6 schematically shows a method of using the filter in accordance with illustrative embodiments. It should be noted that the process of FIG. 6 is a simplified version of a more complex process of using the filter. As such, the actual process may have additional steps that are not discussed. In addition, some steps may be performed in a different order, or in parallel with each other. Accordingly, discussion of this process is illustrative and not intended to limit various embodiments of the invention. Those skilled in the art therefore can modify the process as appropriate.

In various embodiments, a method of using the filter described begins at step 602 by providing a mask 10 that includes the UVC durable filter 16. The filter 16 may be formed from the various materials described herein. The filter 16 may be UVC transmissive and/or UVC diffusively reflective to assist with dosing UVC throughout the entirety of the filter 16. The process then proceeds to step 604, where the user breathes through the mask so that air is filtered by the filter 16. This may be accomplished by a user placing the mask on their face and breathing through the filter. The process then proceeds to step 606, which radiates the filter with UVC. As described previously, the mask may include LEDs. The LEDs 100 may be constantly or periodically dosed. Additionally, or alternatively, the filter 16 may be removable from the mask, and it may be removed and disinfected using UVC. The process then proceeds to step 608, which reuses the mask. In some embodiments, the filter is placed back in the mask. The user puts the mask over their face and continues to breath normally with the mask.

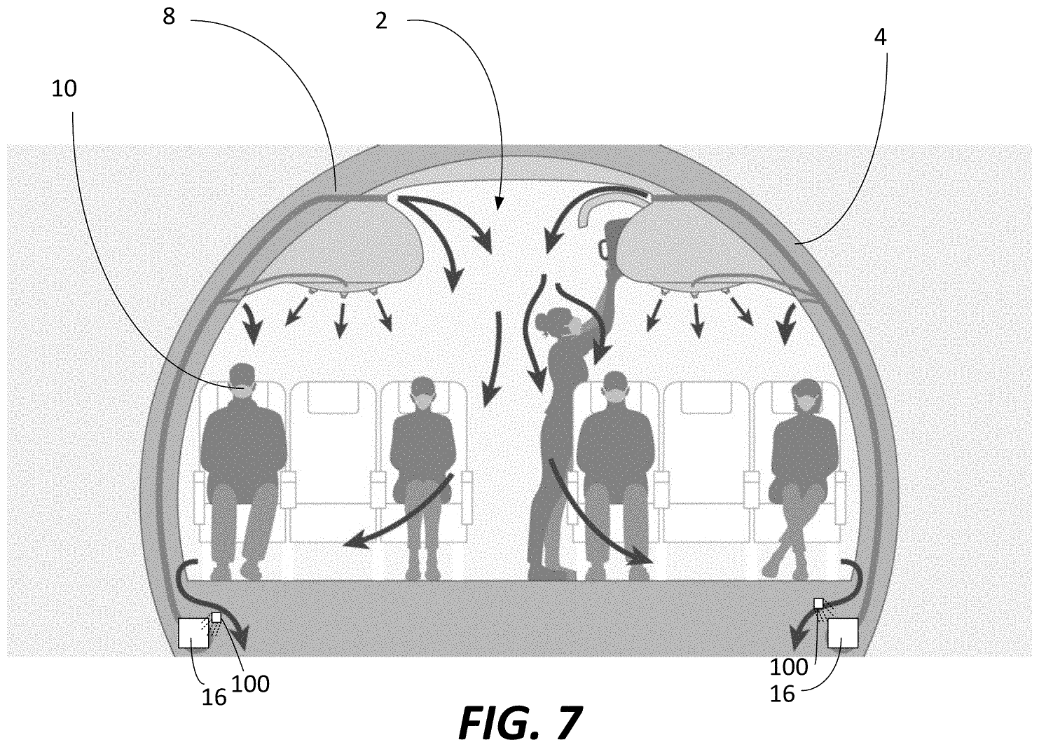

FIG. 7 schematically shows the filter 16 in use a cabin 2 of a commercial jet aircraft 4 in accordance with illustrative embodiments of the invention. Given the current outbreak of Coronavirus Disease 2019 (COVID-19) caused by the novel coronavirus SARS-CoV-2, there is renewed interest in reducing risk of infection in enclosed spaces. General safety recommendations include wearing masks 10, such as cotton masks, disposable masks, N95 masks, and/or elastomeric masks. However, masks 10 work with varying degrees of efficiency. For example, N95 masks 10 capture about 95% of particles that are 0.3 microns or greater. Even so, masks 10 are generally not as efficient as an air filtration system of the aircraft 4.

The air filtration system assists with refreshing air in the cabin 2. Air volume in the cabin 2 of a commercial aircraft 4 is generally refreshed every two to four minutes. For example, as shown in FIG. 7, air 8 flows into the cabin 2 vertically—it enters from overhead vents and is sent downward, exiting at floor level. After the air 8 leaves the cabin 2, about half is discharged outside the aircraft 4 (depending on the make and model), and the rest is recirculated throughout the cabin 2. Fresh air 8 is also brought into the aircraft 4 and circulated around the cabin 2.

Generally, the recirculated air 8 passes through the air filtration system, which frequently includes a High Efficiency Particulate Air (HEPA) filter. HEPA filters are about 99.7% effective at capturing microbes, dust, and particulates down to 0.3 microns. HEPA filters include a mix of filaments and fibers that carry a static charge that attracts various microbes and particles. As the particles travel through the air filtration system, they are captured and retained within the HEPA filter. Various embodiments may include a UVC-durable and UVC-transmissible filter 16, as described throughout the specification.

Over time, with enough volume or use, pathogens eventually separate and penetrate the filter due to their sub-micron size. The SARS-CoV-2 virus is approximately 0.125 micron or 125 nanometers in diameter. However, it often travels in biological aerosols (e.g., from coughing or sneezing) that range in size from 0.5 micron −3.0 micron. These aerosols are largely captured by the filter 16, but it is possible that the virus/pathogen may separate from the aerosol if, for example, the aerosol evaporates.

As the filter 16 becomes clogged with moisture, aerosols eventually evaporate or push through the filter 16. Therefore, illustrative embodiments disinfect the filter 16 as the pathogen is trapped thereon. Illustrative embodiments may be decontaminated using UVC (e.g., from the LED 100) because the filter 16 is transparent to UVC, and also UVC durable (e.g., not vulnerable to degradation by UV0C). Thus, the filter 16 does not provide “dark areas” where the pathogens are protected from UVC disinfection. Illustrative embodiments provide a system including a chamber with the filter 16 that may be reliably and durably disinfected using UVC radiation.

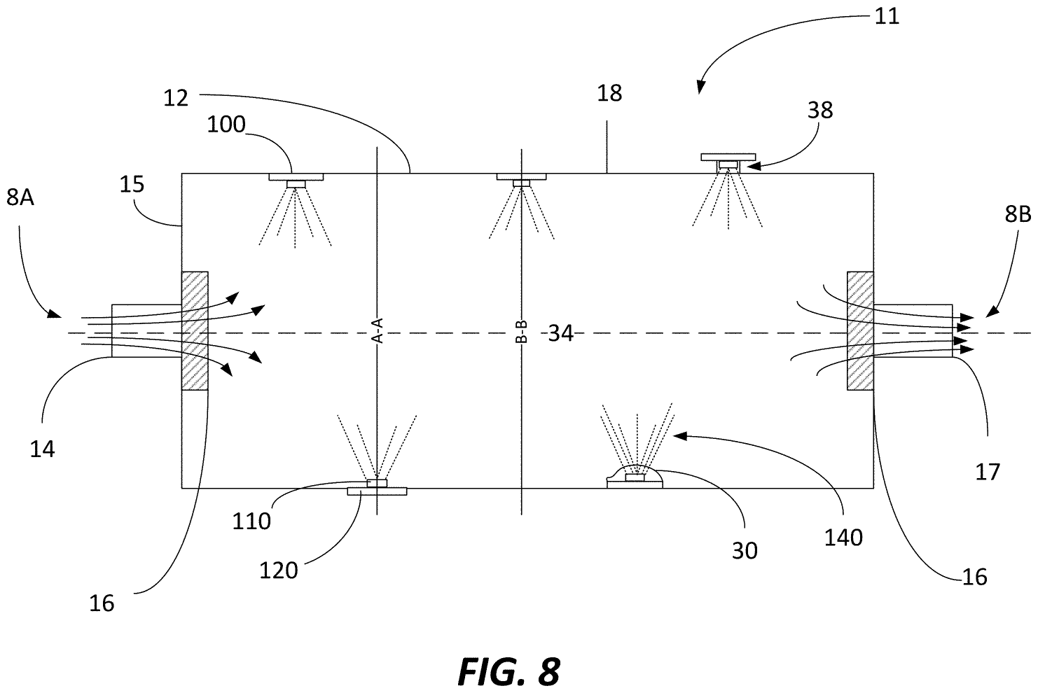

FIG. 8 schematically shows the filter 16 in use with a UVC photoreactor 11 in accordance with illustrative embodiments of the invention. Contaminated air 8A enters the chamber 12 of the reactor 11 through an inlet 14. The contaminated air 8A is disinfected (partially or completely) in the chamber 12 and exits as disinfected air 8B from an outlet 17 of the chamber 12. For example, the outlet 17 may be directly in fluid communication with the cabin 2 of the airplane, an automobile, or an elevator. Additionally, the outlet 17 may be upstream of a traditional air filtration system.

The reactor 11 may be used to disinfect large quantities of air, such as the air 8 circulating in the cabin 2. To assist with passing adequate volumes of air 8 in a timely manner, illustrative embodiments may include a fan or a pump (not shown) fluidly coupled, upstream and/or downstream, of the chamber 12.

Illustrative embodiments position the LEDs 100 in the chamber 12 such that one or more of the LEDs 100 face a sidewall 18 of the chamber 12 (e.g., light emitting surface of the chip 110 faces perpendicular to a longitudinal axis 34 of the chamber 12), as shown in FIG. 2. By facing the sidewall 18, UVC radiation 140 is substantially directed towards the highly reflective sidewall 18. However, some embodiments may additionally, or alternatively, position LEDs 100 so that they face along the length of the chamber 12 (e.g., facing parallel to the longitudinal axis 34 of the chamber 12). Positioning LEDs 100 so that they are facing perpendicular to the longitudinal axis provides a number of advantages when disinfecting air (e.g., particular large volumes of air). Furthermore, various embodiments may include a light pipe 38 that couples the LED 100 with the inside of the chamber 12.

Inside of the chamber 12 are one or more filters 16 configured to trap pathogens of varying sizes. The filter 16 may be positioned immediately adjacent to the inlet 14 and/or the outlet 17. Positioning the filter 16 immediately adjacent to the inlet 14 provides a number of advantages. For example, droplets/aerosols are prevented or inhibited from making it into the chamber 12 where they would otherwise stick to the walls 18 and coat the inside of the chamber 12. Furthermore, the introduction of dust into the chamber 12 is prevented or hindered. Dust undesirably may coat and reduce the reflectivity of the walls 18.

Although FIG. 8 shows the filter 16 immediately adjacent to the inlet 14 and the outlet 17, the filter 16 may be positioned in a variety of ways within the chamber 12. For example, the filter 16 may be substantially perpendicular to the longitudinal axis 34 at any position inside the chamber 12. Preferably, the filter 16 spans the entire diameter/width of the chamber 12, such that there are no zones where air 8 can bypass the filter 16. In various embodiments, the filters 16 are configured to capture particles at least as small as the aerosols that carry the SARS-CoV-2 virus. When pathogens are trapped in the filter 16, UVC radiation 140 emitted by LEDs 100 inactivates and/or kills the pathogens.

As described above, illustrative embodiments prevent or inhibit droplets/aerosols from making it into the chamber 12 (e.g., as these droplets release virus as the droplets evaporate). It is preferable to kill the virus while it is captured in the filter 16. Therefore, various embodiments may radiate the filters 16 with LEDs 100 (e.g., by direct transmission or indirectly by reflection). In some embodiments, one or more LEDs 100 may be embedded in the filter 16. Some virus may still make it through the filter 16, but the LEDs 100 in the chamber 12 disinfects a large proportion of virus that makes it through the filter and into the chamber 12. To that end, at least some of the LEDs 100 are between filters 16, and/or downstream of the filter 16 nearest the inlet 14. Positioning the LEDs 100 on the sidewall 18 rather than on the end wall 40/entrance also allows the UVC radiation to disinfect pathogens without having to pass radiation through the filter 16.

In various embodiments, the chamber 12 is configured to assist with disinfection. For example, the chamber may have an elongated length of about 1 meter. Some embodiments may form the inner walls 18 and/or 40 of the chamber 12 from an inexpensive material that is about 70% reflective to UVC, such as aluminum. Additionally, or alternatively, the walls 18 and/or 40 may be coated with a highly UVC reflective material, such as PTFE.

Some embodiments may include a lower UVC reflective material (e.g., 70% UVC reflective aluminum). This advantageously reduces costs relative to highly UVC reflective material, but require the introduction of a larger fluence of UVC radiation compared to higher UVC reflective material, all else being the same. Therefore, some embodiments may use a mercury lamp to provide large UVC power output. However, UVC lamps suffer from a number of disadvantages. For example, UVC lamps are constantly left on to avoid the stresses of thermal cycling and/or to prevent degradation of the output power of the lamp. However, uninterrupted use of the lamp may waste energy and reduce the useful life of the system. In contrast, the LED 100 can be power cycled, and turned on and off instantly. Furthermore, mercury lamps emit light in 160 degrees and generally are positioned in the center of the chamber 12. Mercury lamps negatively impact the overall average reflectivity in the chamber by undesirably functioning as an absorber in the center of the chamber 12.

Accordingly, illustrative embodiments may use LEDs 100 instead of a mercury lamp, greatly increasing the average reflectivity inside the chamber 12 (i.e., because the mercury lamp is no longer a large absorber). In some embodiments, the reflectivity of the sidewalls 18 of the chamber 12 may be high, such as 90% UVC reflective or higher. The high reflectivity causes the emitted radiation to have a large total path length before absorption (e.g., because of a large number of reflections and because of the low UVC absorption of air). Thus, although the diameter of the chamber 12 is small (e.g., decimeter scale), the total path length of the emitted light is large (e.g., kilometer scale). The high reflectivity of the chamber 12 allows the LEDs 100, which in general have a lower power output than the mercury lamp, to provide up to a 1-log reduction in pathogens. Therefore, the fluence inside the chamber 12 is considerably high despite the relatively low power output of LEDs 100 (as compared to mercury lamps).

Some embodiments may use mercury lamps, however, mercury lamps do not provide for directing light emission in the way LEDs 100 do. It was thought in the art that mercury lamps are preferable over LEDs 100 because LEDs 100 were considered not to be strong enough (i.e., because of the low reflectivity of the walls 18). Therefore, the practice is the art is to aim the light beams parallel to the longitudinal axis 34, as opposed to perpendicular. However, illustrative embodiments advantageously emit light in a direction perpendicular to the longitudinal axis 34. Furthermore, the state of the art using mercury lamps fails to disclose the advantages of a large chamber 12, because the light is not directed (e.g., mercury lamp just scatters), mercury lamps achieve a very low reflectivity.

Furthermore, as compared to water, air has a considerably lower UVC absorption coefficient. As a result, the ultimate path length of UVC within the high reflectivity chamber may be on the scale of kilometers (as opposed to centimeters when disinfecting water). Furthermore, air does not need to achieve the same degree of disinfection in one pass through the chamber as is usually achieved for water. Generally, water disinfection applications seek to achieve about a 3-log reduction in pathogens. In contrast, for air quality applications, a 50% reduction to 90% reduction (1-log) may be achieved for a single pass through the chamber. In contrast to water disinfection applications, the air 8 passes through the filter 16 repeatedly as it is recirculated (as opposed to being drank by a user). Therefore, the air 8 is repeatedly disinfected in the chamber 12. This greatly improves the probabilities of avoiding the spread of disease, as the likelihood of getting sick generally has some proportionality to the size of the virus load.

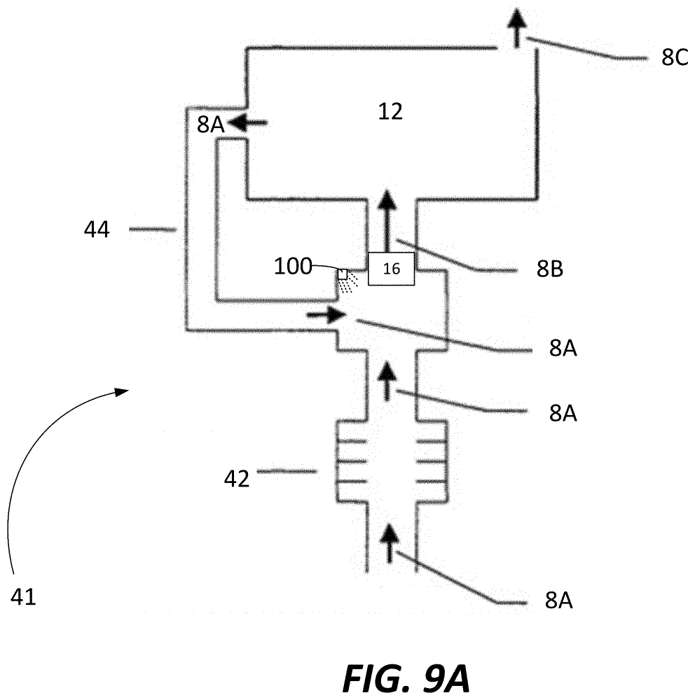

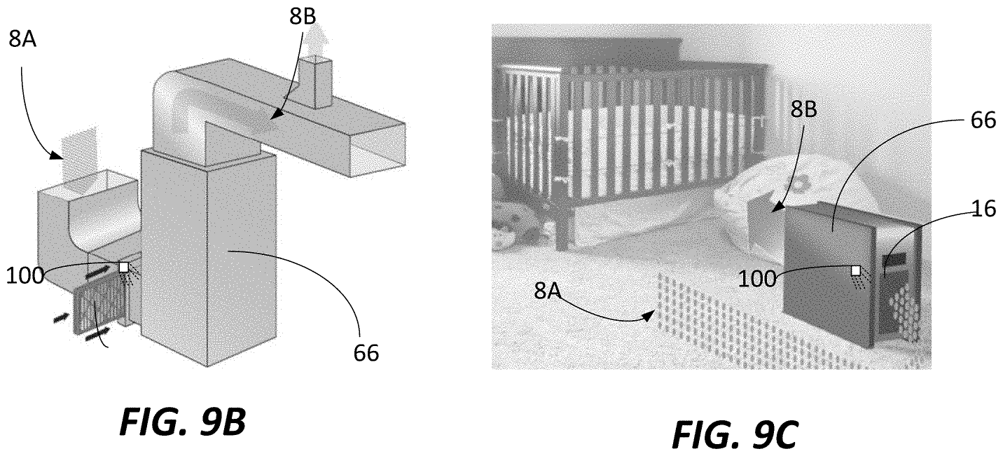

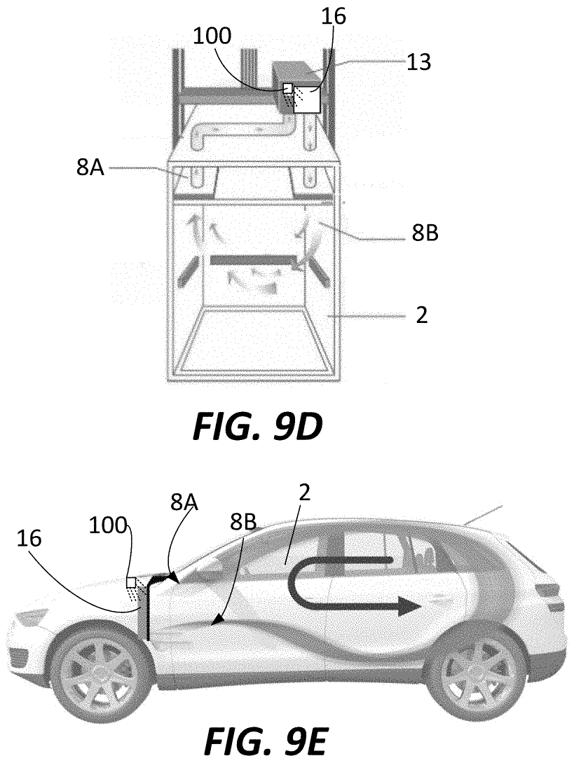

FIGS. 9A-9E schematically show the filter 16 in various use scenarios in accordance with illustrative embodiments of the invention. For example, FIG. 9A schematically shows a diagram of the filter 16 coupled coupled with an environmental control system 41 of the aircraft 4, in accordance with illustrative embodiments of the invention. In some embodiments, the reactor 10, and thus the chamber 12, may be mounted downstream of an aircraft AC system 42, so that the cold air 8A cools the LEDs 24 in the reactor 10.

Air 8A from the cabin 12 may be recirculated via recirculation channel 44 prior to being passed through the reactor 10, and coming out as disinfected air 8B. In a similar manner, air 8A from outside of the aircraft 4 may enter the AC system 42, and then make its way through the reactor 10. As shown in FIG. 6, some of the disinfected air 8B goes into the cabin and is again recirculated as air 8A. However, some other portion of the air 8C may be removed from the aircraft 4.

In addition to commercial aircrafts and vehicles, illustrative embodiments may implement the filter 16 in a variety of settings, including residential settings. For example, the filter 16 may be positioned within an HVAC system (e.g., FIG. 9B). To that end, the filter 16 may be sized in traditional HVAC filter sizes that are commonly available for home use. In various embodiments the filter 16 may be implemented in a portable air cleaning device 66 (e.g., a home filter device) as shown in FIG. 9C. In both FIGS. 9B and 9C, the LED 100 and the filter 16 may be positioned internal to the HVAC system 64 and/or the portable air cleaning device 66.

FIG. 9D schematically shows the filter 16 being used to filter air in the cabin 2 of an elevator in accordance with illustrative embodiments. The LED 100 and/or the filter 16 may be integrated into the air circulation system of the elevator. For example, the LED 100 and the filter 16 are shown integrated in the air conditioning unit 13 of the elevator. FIG. 9E schematically shows the filter 16 being used to filter air in the cabin 2 of a car in accordance with illustrative embodiments. Similar to FIG. 9D, the LED 100 and the filter 16 may be integrated into the air circulation system of the vehicle.

In various embodiments, the LED 100 may be operated on a periodic schedule.

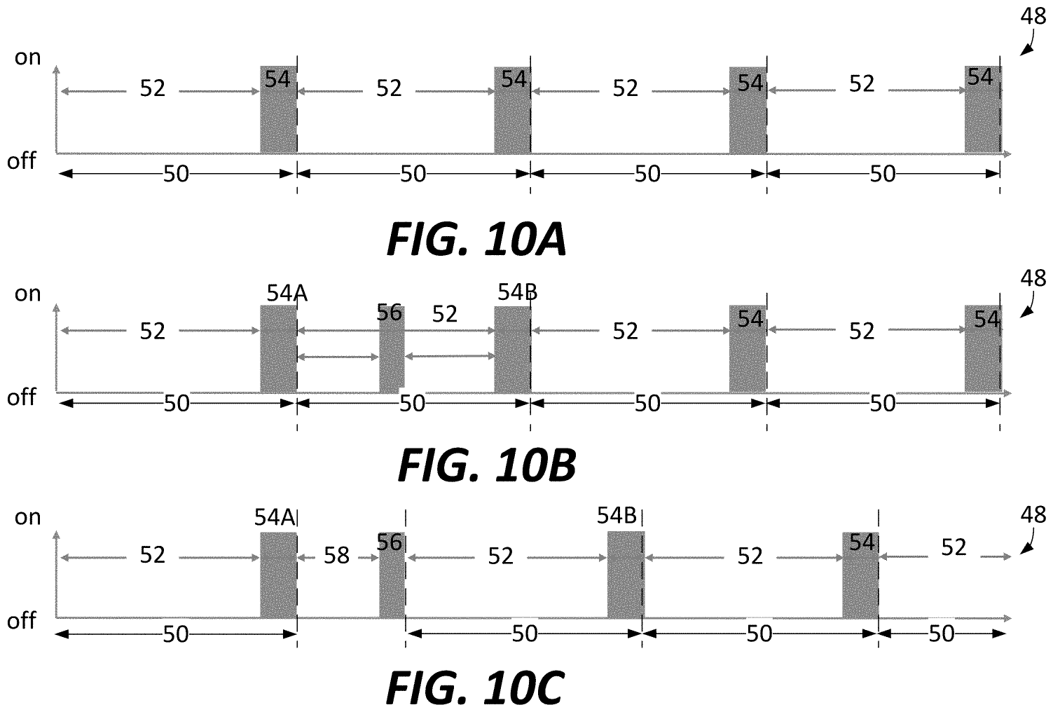

FIG. 10A schematically shows a periodic dosing protocol 48 in accordance with illustrative embodiments of the invention. As shown, the schedule 48 includes a plurality of recurring periods 50. Although shown substantially identical, each of the periods 50 may differ. However, preferred embodiments have substantially identical periods 50.

As described previously each cycle has the active duration 54 and the inactive duration 52. The ratio of the active duration 54 relative to the period 50 is known as a duty cycle. Illustrative embodiments have a duty cycle (active duration 54:duration of period 50) of less than 1:60, for example, 1:100, 1:200, 1:400, 1:1440, 1:2880, or 1:5760. Here, “less than” or “smaller” duty cycle means that the active duration 54 is shorter relative to the period 50. The relatively small duty cycle provides many of the advantages previously described regarding energy savings. Additionally, the LED 100 can instantly power on and off, allowing for short active durations 54.

In illustrative embodiments, the lower limit for filter 16 disinfection is approximately 10 seconds every 12 hours (e.g., duty cycle of 1:4348). Additionally, in illustrative embodiments, the upper limit may be around 2 minutes every hour (e.g., duty cycle of 1:30). However, in some other embodiments, the upper limit may be a duty cycle of 1:1. In some embodiments, the LED 100 may be cycled on when a blower is turned on. Various embodiments may use a heat sink for short term operation (e.g., 10 second operation) and for longer time operation may use pulse mode operation and air flow for cooling. Various embodiments may dose at least 2 mJ/cm² of UVC radiation for 1 log removal value (LRV) of SARs COV-2. In various embodiments, this dosage may be achieved with about 20 seconds of radiation.

In some embodiments the active duration 54 may be greater than about 1 second, about 30 seconds, about 1 minute, or about 5 minutes. The active duration 54 may also be less than about 5 minutes, or about 10 minutes. The time of the period 50 may be between about 30 minutes and about 48 hours. Preferably, the period 50 is less than about 24 hours, to reduce the likelihood that colonies 42 have time to attach to a surface and begin to proliferate. Additionally, in some embodiments the period 50 is greater than about 1 hour, to provide reduced power usage.

The process then proceeds to step 506, which activates the LED 100. As mentioned earlier, one or more LEDs 100 may be activated. For simplicity, a single LED 100 is discussed here. The LED 100 may be pulsed as a single impulse (e.g., instantaneously power on and then off). However, it is contemplated that for the level of UVC radiation required to disinfect the various pathogens trapped in the filter 16, that the LED 100 is dosed for some duration 10. In some embodiments, a plurality of LEDs 100 may be activated each period 50. Some other embodiments may alternate to a different LED 100 for each period 50.

In some embodiments, the LED 100 may be constantly powered on. However, constant powering of the LED 100 may lead to an undesirable user experience, degradation of quality of UVC radiation, unnecessary power expenditure, and thermal management problems. When the LED 100 emits UV light, it can produce a considerable amount of heat. Undesirably, excess heat negatively impacts the light output and lifetime of the LED 100. Thus, proper thermal management preferably keeps the junction temperature (TJ) as low as is required for the given application and maintains the performance of the LED. The word “junction” refers to the p-n junction within the LED 100 die, where the photons are generated and emitted. Heat may be transferred away from this junction to the ambient by coupling a heat sink with the LED 100. To further assist with heat transfer, illustrative embodiments preferably dose the LED 100 in periods 50.

In some embodiments, a trigger may activate the LED 100. As described here, the trigger does not include the normally scheduled activation period 54 of the periodic dosing schedule 48. In various embodiments, the trigger may include the number of breaths taken by the user, a volume of filtered air, or a set amount of time since the last disinfection. Additionally, or alternatively, the LED 100 may be triggered by the user through their smartphone (e.g., by sending a signal through a remote access module). Additionally, or alternatively, a sensor may determine that pathogens levels in the filter 16 have reached a particular trigger threshold, and trigger activation of the LED 100.

When the trigger is activated or the periodic dose is scheduled, an LED control module sends a signal to one or more of the LEDs 100 that causes them to transmit UVC radiation into the photoreactor 11 (e.g., into the main photoreactor zone 110). The LEDs 100 may have an active duration 54 that lasts for the entire period that the user is breathing, or alternatively, may have a set activation duration 54 upon detection of the trigger (e.g., activate LEDs 100 for ten seconds from trigger).

When a periodic dose is requested (e.g., by a periodic dosing module), then the LED 100 is activated for the prescribed duration 54. In some embodiments, the periodic dosing may include a more complex period 50 (e.g., 2-minute activation duration 54 at the 12-hour mark, 1-minute activation duration 54 at the 24-hour mark). However, in some other embodiments, the dosing schedule 48 may be irregular or triggered by some other event (e.g., pathogen 42 count in filter 16 surpasses a trigger level).

When dosed, the LED 100 is activated for the length of the activation duration 54. The length of the LED activation duration 54 may be predetermined and set by a microcontroller. For example, a scheduled off-time 52 may expire before sending a signal to the LED 100 to begin the LED activation duration 54. In various embodiments the LED 100 may communicate with a timer to accurately determine when to begin the activation duration 54. After the expiration of the activation duration, the timer begins counting the amount of time since the LED 100 was last dosed.

Thus, the off-time 52 until the onset of the next active duration 54 is determined based on the time the LED 100 was last dosed. However, some embodiments determine the off-time 52 based on the expiration of the scheduled active duration 54.

FIG. 10B schematically shows a dosing schedule 48 with periods 50 based on expiration of the last scheduled UVC dose in accordance with illustrative embodiments of the invention. Thus, if the trigger is detected, and the LED 100 is trigger activated 56 outside of the dosing schedule 48 (e.g., by a user trigger via smartphone), the next scheduled active duration 54B is based off the expiration of the previously scheduled active duration 54A. Thus, some embodiments maintain the scheduled period 50 regardless of how often the LED 100 is activated.

FIG. 10C schematically shows an adjusted dosing schedule 48B with periods 50 based on expiration of the last UVC dose in accordance with illustrative embodiments of the invention. Thus, if the LED 100 is trigger activated 56 outside of the dosing schedule 48 (e.g., by a user trigger via smartphone), the next scheduled active duration 54B is based off the expiration of the most recent LED activation 56 (i.e., even if it is unscheduled). Accordingly, some embodiments restart the period 50 based on the most recent LED 100 activation.

As shown, the active duration 54 may occur at regular intervals. For example, the active duration 54 may be initiated for 2-minutes of a 12 hour period 50. This may occur on a repeated basis. Thus, another active duration 54 that lasts for 2-minutes occurs at the 12-hour mark, and then again at the 24-hour mark. In such embodiments, the duty cycle is less than 1% (i.e., duty cycle of less than 1:100 (active duration 54:total time per cycle 50)). Indeed, in the above described example, the duty cycle is less than 0.5%. The small duty cycle results in treatment of the water 22 (e.g., disinfection), considerable power savings, and greatly extends the useful life of the LED 100.

The periodic dosing module 118 may request a dose based on the preset timer 122. For example, as shown in FIG. 10A, the dose may automatically be requested automatically after some inactive period 52. Thus, in some embodiments, the dose may be delivered (active duration 54) automatically after a preset amount of time. However, in some other embodiments, the inactivate period 52 may be adjusted based on, e.g., the period of time since the LED 100 was activated by remote request, the number of pathogens within the filter 16, and/or the amplitude of the output power of the LEDs 100.

In a similar manner, the LED active duration 54 may automatically begin after each inactive period 54. As described previously, the temporal length of the active duration 54 may be preset by the microcontroller. In some embodiments, the active duration 54 may be the same length for repeated doses. However, in some other embodiments, the activation period 10 may be adjusted, for example, based on the volume of water 22 within the reactor 11, the period of time since the LED 100 was activated through air flow or remote request, the volume of air within the reactor 11, and/or the amplitude of the output power of the LEDs 100. Thus, as an example, the activation period may be shorter for a stronger dose of UVC or longer for a weaker dose of UVC.

In some embodiments, the periodic dosing schedule may be set to activate the LEDs 100 for 2 minutes (e.g., continuously) every 12 hours. As another example, the LEDs 100 may be active for 1 minute (e.g., continuously) every 6 hours. In some embodiments, the LEDs 24 may provide a dosage of, for example, 5 mJ/cm², for example, for 50 second active duration 54. In some other embodiments, the LEDs 100 may provide a dosage of 12.5 mJ/cm² for 125 second active duration 54. In various embodiments, dosages of between about 5 mJ/cm² and about 12.5 mJ/cm² may be provided during the active period 54 for about 10 seconds to about 10 minutes. However, various embodiments may use smaller dosages and/or shorter time frames than listed above. In some embodiments, the various dosage times and mJ/cm² values listed above may be considered to provide an upper limit on a range of dosages. Illustrative embodiments reduce the concentration of pathogen trapped in the filter 16 to a small number so that a person breathing air filtered through the filter 16 does not become sick. Various embodiments are configured to radiate the filter 16 using UVC to achieve at least 1 LRV of pathogen, which is a 90% reduction in pathogen.

While illustrative embodiments here have referred to use in masks, it should be understood that the advantages of the invention can be achieved in any variety of types of filter systems. For example, illustrative embodiments may be used to filter airplane or automobile cabins, HVAC systems, a variety of enclosed spaces intended to have human occupancy (e.g., elevator), or other air management devices. This would have advantage over UVC photoreactors that do not use such a filter, as the filter could trap airborne aerosols, allowing for an effectively longer residency time in the photoreactor, and therefore increased dose of UVC.

As used in this specification and the claims, the singular forms “a,” “an,” and “the” refer to plural referents unless the context clearly dictates otherwise. For example, reference to “an LED” in the singular includes a plurality of LEDs, and reference to “the filter” in the singular includes one or more filters and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A system comprising:
a filter configured to capture aerosol particles, the filter formed of a material that is UVC durable and UVC transmissive; and
a UVC LED configured to emit UVC radiation into the filter.

2. The system of claim 1, wherein the filter is formed of a plurality of fibers.

3. The system of claim 1 further comprising a plurality of LEDs configured to emit UVC radiation towards the filter.

4. The system of claim 1, wherein the filter is integrated into an HVAC system.

5. The system of claim 1, wherein the filter is integrated into a portable air cleaning device.

6. The system of claim 1, wherein the filter is integrated into an air circulation system of an aircraft, a vehicle, or an elevator.

7. The system of claim 1, wherein the filter is integrated into a mask configured to fit around a face of a user.

8. The system of claim 7, further comprising a UVC absorbent blocker configured to protect a portion of a user's face from UVC.

9. The system of claim 8, wherein the UVC absorbent blocker includes UVC goggles.

10. The system of claim 1, wherein the filter is formed from at least one of PTFE, PET, and glass.

11. The system of claim 1, wherein the LED is integrated into the filter.

12. The system of claim 1, wherein the filter is formed of UVC translucent material.

13. The system of claim 1, further comprising a plurality of filters.

14. The system of claim 1, wherein the LED is configured to be periodically dosed by a controller, the controller configured to set a duty cycle for the period, wherein the duty cycle is a ratio of the active duration to the period, wherein the duty cycle is less than or equal to about 1:100.

15. The system of claim 1, wherein the duty cycle is between about 1:100 and about 1:5760.

16. The system of claim 1, wherein the active duration is between about 10 seconds and 5 minutes.

17. The system of claim 1, wherein the period is between about 30 minutes and 48 hours.

18. The system of claim 1, wherein the LED is a lidless type LED.

19. An apparatus comprising:
a filter formed from a material that is UVC transmissive and UVC durable, the filter having pores through which air may travel, the filter configured so that radiation from a UVC LED irradiates the entirety of the filter to inactivate pathogens captured by the filter.

20. The apparatus of claim 19, wherein the filter is formed from PTFE, PET, and/or glass.

21. The apparatus of claim 19, wherein the filter is configured to filter the cabin of an airplane or an automobile.

22. The apparatus of claim 19, further comprising thermal insulation configured to protect the user's face from heat generated by the LED.

23. A method of filtering air, the method comprising:
providing a filter configured to capture aerosol particles, the filter formed of a material that is UVC durable and UVC transmissive;
providing a UVC LED configured to emit UVC radiation into the filter;
capturing a pathogen using the filter; and
radiating the filter with UVC light to disinfect the filter.

24. The method of claim 23, wherein the UVC light reaches all portions of the filter.

25. The method of claim 23, wherein the UVC light is periodically dosed for at least 5 seconds to radiate the filter with at least 5 mJ/cm² of UVC.

26. The method of claim 23, wherein the filter is configured to be dosed with UVC for at least 1000 hours without degrading.

27. The method of claim 23, wherein the pathogen captured in the filter is disinfected by at least 1 log reduction value.
